(19) United States
(12) Patent Application Publication
(10) Pub. No.: US 2011/0274678 A1
(43) Pub. Date: Nov. 10, 2011

(54) Beverage

(76) Inventor: Kourosh Dibadj, McLean, VA (US)

(21) Appl. No.: 13/102,099

(22) Filed: May 6, 2011

Related U.S. Application Data
(60) Provisional application No. 61/332,365, filed on May 7, 2010.

(51) Int. Cl. A61K 38/43; A61K 31/59; A61K 31/20; A61K 31/70

(52) U.S. Cl. 424/94.1; 514/52; 514/168; 514/560

(57) ABSTRACT

A nutritional beverage composition for providing nutrition to a patient suffering from a kidney ailment comprises a flavor component and a natural sweetener, and is further characterized in that it does not contain amounts of particular ingredients that may not be desirable in treating kidney ailments. In one embodiment of the present invention, the beverage composition lacks substantially any potassium, phosphorous, magnesium, high fructose corn syrup, vitamin A, vitamin K, or vitamin E.

The present application claims benefit and priority of U.S. Application Ser. No. 61/332,365, filed May 7, 2010 and entitled “Beverage”, the disclosure of which is incorporated herein by reference.

### FIELD OF THE INVENTION

The present invention relates to a packaged beverage providing hydration and nutritional benefits to those who suffer from various kidney ailments.

### BACKGROUND OF THE INVENTION

Patients with kidney disease have limited options when choosing among commercially available beverages due to the presence of ingredients that are known to be harmful to people with damaged or malfunctioning kidneys.

The kidney is an organ that provides several functions, including filtering and removing waste products, balancing the body's fluid content, producing hormones that help control blood pressure and make red blood cells, and activating vitamin D for healthy bones. If damaged, kidneys are less effective at removing the waste products from the blood. As a result, the waste products continue to build up in the bloodstream. Further, if the kidneys are damaged and unable to produce the same levels of hormones as a healthy kidney, the bone marrow is not as effective at producing red blood cells, which can result in anemia.

In the long run, damaged kidneys can cause a wide variety of problems, but because the damage may not be seen or felt right away, many people with damaged kidneys may not realize they have such health issues until they are treated and start feeling better.

Patients with failed or failing kidneys can be treated using a combination of diet, medication and dialysis. Diet can help in reducing the levels of different types of waste products, while reducing the severity of many of the symptoms of kidney disease. Because kidney patients can experience a loss of appetite as part of their symptoms, it is important for patients and their doctors to be aware that such a symptom can result in poor nutrition, as well as the need for dialysis and supplement drinks.

Unfortunately, currently available packaged beverages include many ingredients that are harmful to kidney patients while not including ingredients that could be helpful to kidney patients. Salt, for example, can increase blood pressure, so beverages high in sodium must be avoided. Potassium is a mineral found in body tissue cells, but too high of a potassium level is bad for the heart. Similarly, a build-up of magnesium is bad for the body. While any excess potassium or magnesium would normally be removed by the kidneys, this may not be the case for a patient with kidney disease.

Phosphates are widely found in foods, and work very closely with the mineral calcium. A healthy body keeps these two substances in balance. High phosphate levels may be associated with itch and can damage blood vessels over time. Damaged kidneys can struggle to maintain a healthy balance of phosphates and calcium; thus, phosphates are generally avoided according to the beverage of the present invention.

### SUMMARY OF THE INVENTION

The present invention provides nutritional beverage compositions used for treating patients with failed or failing kidneys. The nutritional compositions can be provided in powder form, for example, and can then be mixed with water to form a nutritional beverage in accordance with the present invention. The beverage of the present invention not only avoids the use of ingredients that can be harmful to those with damaged, failing or malfunctioning kidneys, but it includes helpful supplements combined in a novel way.

In a preferred embodiment, the beverage of the present invention contains a flavor component and a sweetener, neither of which contains substantially any potassium, phosphorous or magnesium, and neither of which contains any sodium. However, the minerals potassium, phosphorous and magnesium are not contained due to their potential to build up abnormally in kidney patients. In one embodiment of the present invention, only a minimal amount of salt is present for taste purposes. In another embodiment of the present invention, no salt is provided due to the potential to increase hypertension and negatively affect kidney function. Further, since long term effects of artificial sweeteners on kidney function and general health is not clear, the beverage of the present invention contains no artificial sweeteners. Due to possible association of high fructose corn syrup (HFCS) with kidney disease and high uric acid levels, a preferred embodiment of the beverage of the present invention contains no HFCS. Additionally, vitamins A, K and E are not provided in a preferred embodiment of the beverage of the present invention, because these vitamins can build up to toxic levels in patients with kidney dysfunction (particularly patients who are on dialysis). While it is desirable to eliminate the foregoing agents entirely, those skilled in the art will recognize that the addition of small or trace amounts of these excluded agents insufficient to harm the patient will be considered encompassed by the present invention.

The compositions of the present invention may comprise numerous vitamins and minerals that have been found to help patients with kidney disease. The vitamins included in the compositions of the present invention may include, for example, vitamin C, vitamin D, vitamin B₁, vitamin B₂, vitamin B₃, vitamin B₅, vitamin B₆, vitamin B₇ vitamin B₉, and vitamin B₁₂. The minerals included in the compositions of the present invention may include calcium and iron, for example. The beverage of the present invention may contain up to 80 kilocalories per one liter (80 kCal/L) serving, and in another embodiment contains no calories. When provided, the calories do not come from fat or high fructose corn syrup.

Since Diabetes Mellitus (sugar diabetes) is a common cause of kidney disease, the amount of sugar (sucrose) will be limited in a preferred embodiment of the beverage of the present invention. In one embodiment of the present invention, xylitol, a natural sugar alcohol sweetener with fewer calories and potentially safer in diabetic patients, may be used as sweetener in the beverage. In another embodiment of the invention, stevia, a natural sweetener, with no calories and potentially safer for diabetic patients, may be used as sweetener for the beverage of the present invention.

Since cardiovascular disease is a major cause of death in patients with kidney failure (and most common cause of death in dialysis patients), the beverage can contain, in one embodiment of the present invention, Omega-3 fatty acids (a-linolenic acid (ALA), docosahexaenoic acid (DHA), and eicosapentaenoic acid (EPA)), as these ingredients can have beneficial effects for heart disease.

It will be appreciated that different combinations of ingredients employed in the different embodiments of the present invention disclosed herein will result in different costs, taste and effect based on the different ingredient compatibilities. For example, providing a range for vitamin C up to 120 mg/L allows for the present invention to accommodate lower or higher concentrations of vitamin C depending upon the individual needs and preferences of the consumer. Additionally, natural or artificial flavor components may be added to the composition. However, it is essential that the flavor component does not contain a measurable amount of potassium, phosphorous, magnesium, high fructose corn syrup, vitamin A, vitamin K or vitamin E.

It will thus be seen from the present disclosure that the nutritional composition and beverage of the present invention can combine a variety of ingredients using a variety of concentrations to result in a composition and beverage that provides proper nutrients while avoiding other potentially damaging ingredients.

### DETAILED DESCRIPTION OF THE INVENTION

It is understood that the present invention is not limited to the particular compositions described herein, as these may vary. Thus, for example, a reference to a vitamin in the form of a particular compound includes equivalents thereof known to those skilled in the art.

The nutritional composition of a beverage for patients with kidney disease requires a unique formulation because of dietary restrictions as well as changes in metabolism, absorption, and excretion of many vitamins, minerals and electrolytes. In particular, a preferred embodiment of the beverage composition includes a flavoring component, a natural sweetener and does not contain potassium, phosphorous, magnesium or high fructose corn syrup, and contains no or minimal amounts of sodium. Additionally, a preferred embodiment of the composition does not contain vitamins A, K or E which can build up to toxic levels in patients with kidney dysfunction. However, a composition of the present invention comprises numerous vitamins and minerals that have been found to help patients with kidney disease. The flavor component can be natural or artificial flavor, and can be ordered in a variety of flavors (e.g., peach, apple, strawberry, kiwi, pomegranate) from companies known in the art to produce flavor components. However, it is essential that the flavor component added to a composition of the present invention does not contain a measurable amount of potassium, phosphorous, magnesium, high fructose corn syrup, vitamin A, vitamin K or vitamin E.

Due in part to lower calcium intake and abnormal metabolism of calcium and vitamin D, patients with kidney dysfunction require supplemental vitamin D which may be included in the composition of the present invention. The recommended dietary allowances (RDAs) of vitamin D currently range from 400-800 IU depending on age. In one embodiment of the present invention, the nutritional composition contains up to 800 IU (20 mcg) of vitamin D.

In addition to vitamin D, compositions of the present invention may comprise various water soluble vitamins that have been found to help patients with kidney disease, (particularly in patients on dialysis treatment), since through the process of dialysis, water soluble vitamins are cleared from body. Reduced levels of vitamin C, for example, have been observed in patients with kidney dysfunction, most likely as a result of a low-potassium diet and decreased food intake. In one embodiment of the present invention, vitamin C may be provided in the form of ascorbic acid. The vitamin C can, for example, be provided in an amount up to 120 mg.

Additionally, patients with kidney ailments that have been placed on a low protein diet exhibit thiamine (vitamin B₁) as well as riboflavin (vitamin B₂) deficiencies. Thus, in one embodiment of the present invention, up to 3 mg of thiamin (vitamin B₁) has been added to the beverage composition. In another embodiment of the present invention, up to 3.4 mg of riboflavin (vitamin B₂) has been included in the beverage composition.

Niacin (vitamin B₃) is involved in numerous enzymatic reactions and is also required for the synthesis of pyroxidine, riboflavin, and folic acid. A niacin deficiency has been noted in dialysis patients and reduced amounts of niacin have been demonstrated in a low protein diet. Therefore, one embodiment of the nutritional composition of the present invention includes vitamin B₃ in the form of niacin. The beverage composition can include, for example, up to 40 mg of niacin (vitamin B₃).

Low protein diets have also been shown to provide only a minimal amount of pantothenic acid (vitamin B₅). Additionally, a decrease in pantothenic acid plasma levels has been observed in dialysis patients. Therefore, one embodiment of the beverage composition includes up to 20 mg of pantothenic acid (vitamin B₅).

A vitamin B₆ deficiency has also been noted in kidney disease patients undergoing dialysis. Therefore, in one embodiment of the nutritional composition, vitamin B₆ in the form of pyridoxine has been provided. The composition may contain, for example, up to 20 mg of pyridoxine (vitamin B₆).

There is some evidence to suggest that patients with kidney disease and dialysis patients may develop a biotin (vitamin B₇) deficiency. Biotin acts as a coenzyme for a number of carboxylases and so it has an important role in gluconeogenesis, fatty acid metabolism, and amino acid metabolism. In one embodiment of the beverage composition, up to 600 mcg of biotin (vitamin B₇) is included.

Folic acid (vitamin B₉) tends to be low in the diet prescribed to patients with kidney ailments. In one embodiment of the present invention, vitamin B₉ in the form of folic acid may be included in the beverage composition. In another embodiment, the naturally occurring form of folic acid, folate, may be used. Up to 1600 mcg of folic acid may be included in the beverage composition, for example.

Vitamin B₁₂ is necessary for folic acid delivery to tissues. A deficiency of vitamin B₁₂ has been observed in patients with kidney deficiencies and dialysis patients. Thus, in one embodiment of the present invention, up to 480 IU (12 mcg) of vitamin B₁₂ may be included in the nutritional composition.

In addition to the helpful vitamins found in embodiments of the beverage composition, omega-3 fatty acids, specifically docosahexaenoic acid (DHA), eicosapentaenoic acid (EPA), and a-linolenic acid (ALA) have been included in an embodiment of the beverage composition. Since cardiovascular disease is a major cause of death in patients with kidney failure (and most common cause of death in dialysis patients), the beverage composition may contain up to 300 mg of omega-3 fatty acids containing ALA, DHA, and EPA. In another embodiment of the nutritional composition, up to 40 mg of coenzyme Q10 is included in the beverage composition.

Furthermore, since patients with kidney disease have a greater tendency for anemia and iron deficiency, the beverage composition may contain supplemental iron. Up to 4 mg of elemental iron is included in one embodiment of the nutritional composition. Additionally, in another embodiment of the nutritional composition, up to 400 mg of elemental calcium is included in the beverage composition.

The beverage can contain up to 80 kilocalories per one liter (80 kCal/L) serving, and in another embodiment contains no calories. When provided, the calories do not come from fat or high fructose corn syrup. Since long term effects of artificial sweeteners on kidney function and general health is not clear, the beverage will not contain artificial sweeteners. Due to possible association of high fructose corn syrup (HFCS) with kidney disease and high uric acid levels, HFCS will not be used. Since Diabetes Mellitus (sugar diabetes) is a common cause of kidney disease, the amount of sugar (sucrose) will be limited in the beverage. In one embodiment of the present invention, up to 20 grams (4 cal/grams) of sucrose is added to the nutritional composition. In another embodiment of the present invention, xylitol, a natural sugar alcohol sweetener with fewer calories and potentially safer in diabetic patients, may be used as sweetener in the beverage. Up to 20 grams (2.4 cal/gram) of xylitol may be included in the beverage composition, for example. In another embodiment of the invention, stevia, a natural sweetener, with no calories and potentially safer for diabetic patients, may be used as sweetener for the beverage.

In one embodiment, the present invention provides a liquid beverage, comprising: water; vitamin D at a concentration up to 800 IU (20 mcg), inclusive of the end points; water soluble vitamins at concentrations according to Example 1 below, inclusive of the end points; and a natural sweetener; without providing any source of magnesium, phosphorous, phosphoric acid, potassium, sodium, vitamin A, vitamin K, or vitamin E. In another embodiment of the present invention, a powdered concentrate of the beverage may be prepared. One serving size, for example, can be equal to 8 g of powder found in one powder pack. The beverage can then be consumed by mixing the powder with the appropriate amount of liquid (i.e., water). For example, one powder pack can be mixed with 16 ounces of water and this would be considered one serving size. A longer shelf life is expected in the powdered preparation. In another embodiment, a liquid beverage concentrate can be prepared which upon mixing with an appropriate amount of liquid (i.e., water) will prepare the final beverage. In another embodiment, the beverage may be carbonated, either by blending the beverage concentrate with carbonated water or blending the beverage concentrate with water followed by carbonation of the blend.

The invention will be further illustrated by the following non-limiting examples.

### Example 1

The following table (Table 1) shows one embodiment of the concentration of different ingredients in the composition per liter of beverage for the present invention. Even though not listed here, the beverage of the present invention may include ingredients not known to be harmful to the kidneys such as flavors, coloring and other such items. Further, the below embodiment is just one embodiment of the present invention. Thus, it will be appreciated that the differences or ranges provided can lead to other versions of the beverage contemplated in accordance with the present invention.

TABLE 1

| Ingredient | Amount |
|---|---|
| Vitamin C | Up to 120 mg |
| Thiamin, B1 | Up to 3 mg |
| Riboflavin, B2 | Up to 3.4 mg |
| Niacin, B3 | Up to 40 mg |
| Pyridoxine, B6 | Up to 20 mg |
| Folic acid, B9 | Up to 1600 mcg |
| Vitamin B12 | Up to 12 mcg |
| Biotin, B7 | Up to 600 mcg |
| Pantothenic Acid, B5 | Up to 20 mg |
| Vitamin D | Up to 800 IU (20 mcg) |
| Calcium | Up to 400 mg elemental calcium |
| Iron | Up to 4 mg elemental iron |
| Stevia | Up to 2 grams (0 cal/gram) |
| Xylitol | Up to 20 grams (2.4 cal/gram) |
| Sucrose | Up to 20 grams (4 cal/gram) |
| Omega 3 fatty acids containing: | Up to 300 mg |
| DHA/EPA | |
| Coenzyme Q10 | Up to 40 mg |

Examples 2 and 3 show example nutritional facts for one embodiment of the composition in liquid form, as well as nutritional facts for one embodiment of the composition in powder form.

### Example 2

Flavor: Pamagrande-Acai
Nutritional Facts
Serving size 8 fl oz (237 ml)
Serving size per container 2
Calories 12 per serving

| Amount | % Daily Value (per serving) | (per bottle) |
|---|---|---|
| Total Fat 0 g | 0% | 0% |
| Sodium 15 mg | <1% | 1% |
| Total Carbohydrate 5g | 2% | 4% |
| Dietary Fiber 0 g | | |
| Sugar 0 g | | |
| Xylitol 5 g | | |
| Protein 0 g | 0% | 0% |
| Potassium 0 mg | 0% | 0% |
| Phosphorus 0 mg | 0% | 0% |
| Omega 3 (DHA/EPA) 40 mg | 25% | 50% |
| Vitamin C | 25% | 50% |
| Vitamin B1 | 25% | 50% |
| Riboflavin | 25% | 50% |
| Niacin | 10% | 20% |
| Pyridoxine | 50% | 100% |
| Folic Acid | 50% | 100% |
| Vitamin B12 | 50% | 100% |
| Biotin | 50% | 100% |
| Pantothenic acid | 25% | 50% |
| Vitamin D | 10% | 20% |
| Calcium | 10% | 20% |

### Example 3

| Flavor: Peach | |
|---|---|
| Nutritional Facts | |
| Serving size 1 Stick Pack (8g) | |
| Serving size per container 1 | |
| Calories 25 per serving | Calories from Fat 0 |
| Amount | % Daily Value (per serving) |
| Total Fat 0 g | 0% |
| Sodium 0 mg | 0% |
| Total Carbohydrate 7 g | 2% |
| Sugar 5 g | |
| Protein 0 g | 0% |
| Potassium 0 mg | 0% |
| Phosphorus 0 mg | 0% |
| Vitamin C | 35% |
| Thiamin, B1 | 70% |
| Riboflavin, B2 | 70% |
| Niacin, B3 | 35% |
| Pyridoxine, B6 | 50% |
| Folate, B9 | 70% |
| Vitamin B12 | 35% |
| Biotin, B7 | 8% |
| Pantothenic acid, B5 | 55% |
| Vitamin D | 70% |

Not a significant source of saturated fat, trans fat, cholesterol, dietary fiber, vitamin A, calcium, and iron.
Percent Daily Values are based on a 2,000 calorie diet
INGREDIENTS: Sugar, Citric Acid, Natural & Artificial Flavor, Maltodextrin, Stevia Extract (Rebaudioside A), Silicon Dioxide, Flax Seed Oil, Fish Oil, Ascorbic Acid, Yellow 6 Lake, Niacinamide, D-Calcium Pantothenate, Riboflavin, Pyridoxine HCL, Thiamin Mononitrate, Folic Acid, Vitamin D3, Biotin, Cyanocobalamin (Vitamin B12).

In one embodiment of the present invention, an 8 g powder pack contains vitamin D, water soluble vitamins, Omega-3 fatty acids, and a natural sweetener comprising sugar and stevia extract. The omega-3 fatty acids may comprise ALA, DHA, and EPA (e.g., fish oil). The amount of each ingredient, for example, is shown in Example 4 below.

### Example 4

| Ingredient | Amount | |
|---|---|---|
| Vitamin C | 21 | mg |
| Thiamin, B₁ | 1.05 | mg |
| Riboflavin, B₂ | 1.19 | mg |
| Niacin, B₃ | 7 | mg |
| Pyridoxine, B₆ | 1 | mg |
| Folate, B₉ | 280 | mcg |
| Vitamin B₁₂ | 2.1 | mcg |
| Biotin, B₇ | 24 | mcg |
| Pantothenic acid, B₅ | 3.5 | mg |
| Vitamin D | 280 | IU |
| Omega-3 (ALA, EPA, DHA) | 30 | mg |
| Stevia | 0.14 | g |

In another embodiment of the nutritional composition of the present invention, the natural sweetener xylitol has been added to the composition.

### Example 5

| Ingredient | Amount |
|---|---|
| Vitamin C | Up to 120 mg |
| Thiamin, B₁ | Up to 3 mg |
| Riboflavin, B₂ | Up to 3.4 mg |
| Niacin, B₃ | Up to 40 mg |
| Pyridoxine, B₆ | Up to 20 mg |
| Folic acid, B₉ | Up to 1600 mcg |
| Vitamin B₁₂ | Up to 12 mcg |
| Biotin, B₇ | Up to 600 mcg |
| Pantothenic acid, B₅ | Up to 20 mg |
| Vitamin D | Up to 20 mcg |
| Omega-3 fatty acids containing DHA/EPA | Up to 300 mg |
| Xylitol | Up to 20 grams (2.4 cal/gram) |

In another embodiment of the present invention, elemental iron has been added to the beverage composition.

### Example 6

| Ingredient | Amount |
|---|---|
| Vitamin C | Up to 120 mg |
| Thiamin, B₁ | Up to 3 mg |
| Riboflavin, B₂ | Up to 3.4 mg |
| Niacin, B₃ | Up to 40 mg |
| Pyridoxine, B₆ | Up to 20 mg |
| Folic acid, B₉ | Up to 1600 mcg |
| Vitamin B₁₂ | Up to 12 mcg |
| Biotin, B₇ | Up to 600 mcg |
| Pantothenic acid, B₅ | Up to 20 mg |
| Vitamin D | Up to 20 mcg |
| Omega-3 fatty acids containing DHA/EPA | Up to 300 mg |
| Sucrose | Up to 20 grams (4 ca1/gram) |
| Iron | Up to 4 mg elemental iron |

In another embodiment of the present invention, elemental calcium has been added to the beverage composition.

### Example 7

| Ingredient | Amount |
|---|---|
| Vitamin C | Up to 120 mg |
| Thiamin, B₁ | Up to 3 mg |
| Riboflavin, B₂ | Up to 3.4 mg |
| Niacin, B₃ | Up to 40 mg |
| Pyridoxine, B₆ | Up to 20 mg |
| Folic acid, B₉ | Up to 1600 mcg |
| Vitamin B₁₂ | Up to 12 mcg |
| Biotin, B₇ | Up to 600 mcg |
| Pantothenic acid, B₅ | Up to 20 mg |
| Vitamin D | Up to 20 mcg |
| Omega-3 fatty acids containing DHA/EPA | Up to 300 mg |
| Sucrose | Up to 20 grams (4 cal/gram) |
| Calcium | Up to 400 mg elemental |
| | calcium |

In another embodiment of the present invention, coenzyme Q10 has been added to the beverage composition.

### Example 8

| Ingredient | Amount |
|---|---|
| Vitamin C | Up to 120 mg |
| Thiamin, B₁ | Up to 3 mg |
| Riboflavin, B₂ | Up to 3.4 mg |
| Niacin, B₃ | Up to 40 mg |
| Pyridoxine, B₆ | Up to 20 mg |
| Folic acid, B₉ | Up to 1600 mcg |
| Vitamin B₁₂ | Up to 12 mcg |
| Biotin, B₇ | Up to 600 mcg |
| Pantothenic acid, B₅ | Up to 20 mg |
| Vitamin D | Up to 20 mcg |
| Omega-3 fatty acids containing DHA/EPA | Up to 300 mg |
| Sucrose | Up to 20 grams (4 cal/gram) |
| Coenzyme Q10 | Up to 40 mg |

In one embodiment of the present invention, the composition comprises a natural sweetener and a flavor component. The flavor component may be natural or artificial; however, neither the flavor component nor the sweetener contains substantially any potassium, phosphorous, magnesium, high fructose corn syrup, vitamin A, vitamin K or vitamin E.

### Example 9

| Ingredient | Amount |
|---|---|
| Flavor Component | — |
| Sucrose | Up to 20 grams (4 cal/gram) |
| Xylitol | Up to 20 grams (2.4 cal/gram) |
| Stevia | Up to 2 grams (0 cal/gram) |

While the invention has now been described in terms of a preferred embodiment of compositional ranges, the skilled artisan will appreciate the various substitutions, modifications, changes, and omissions may be made without departing from the spirit thereof.

1. A beverage composition for providing nutrition to a patient suffering from a kidney ailment, comprising:
a flavor component substantially free of potassium, phosphorous, magnesium, high fructose corn syrup, vitamin A, vitamin K and vitamin E; and
a natural sweetener substantially free of potassium, phosphorous, magnesium, high fructose corn syrup, vitamin A, vitamin K and vitamin E.

2. The beverage composition of claim 1, wherein the beverage composition is further characterized in that it contains trace amounts of sodium.

3. The beverage composition of claim 1 further comprising nutritionally effective amounts of water soluble vitamins, wherein the water soluble vitamins comprise: vitamin C; thiamin (vitamin B₁); riboflavin (vitamin B₂); niacin (vitamin B₃); pantothenic acid (vitamin B₅); pyridoxine (vitamin B₆); biotin (vitamin B₇); folic acid (vitamin B₉); and vitamin B₁₂.

4. The beverage composition of claim 3, wherein the water soluble vitamins are present in amounts of: vitamin C, up to 120 mg; thiamin, B₁, up to 3 mg; riboflavin, B₂, up to 3.4 mg; niacin, B₃, up to 40 mg; pantothenic acid, B₅, up to 20 mg; pyridoxine, B₆, up to 20 mg; folic acid, B₉, up to 1600 mcg; vitamin B₁₂, up to 12 mcg; and biotin, B₇, up to 600 mcg.

5. The beverage composition of claim 1 further comprising a nutritionally effective amount of vitamin D, wherein the vitamin D is present in an amount of up to 20 mcg.

6. The beverage composition of claim 1, wherein the natural sweetener includes sucrose present in an amount of up to about 20 grams.

7. The beverage composition of claim 1, wherein the natural sweetener includes xylitol present in an amount of up to about 20 grams.

8. The beverage composition of claim 1, wherein the natural sweetener includes stevia.

9. The beverage composition of claim 1 further comprising omega-3 fatty acids in an amount of up to about 300 mg, wherein the omega-3 fatty acids comprise a-linolenic acid (ALA), docosahexaenoic acid (DHA) and eicosapentaenoic acid (EPA).

10. The beverage composition of claim 1 further comprising elemental calcium in an amount of up to about 400 mg.

11. The beverage composition of claim 1 further comprising elemental iron in an amount of up to about 4 mg.

12. The beverage composition of claim 1 further comprising coenzyme Q10 in an amount of up to about 40 mg.

13. The beverage composition as defined by claim 1, said composition being in powder form.

14. The beverage composition as defined by claim 1 further comprising water and being in liquid form.

15. A beverage composition for providing nutrition to a patient suffering from a kidney ailment comprising:
a flavor component substantially free of potassium, phosphorous and magnesium; and
a natural sweetener substantially free of potassium, phosphorous and magnesium.

16. A beverage composition for providing nutrition to a patient suffering from a kidney ailment comprising:
a nutritionally effective amount of vitamin D;
nutritionally effective amounts of water soluble vitamins, wherein the water soluble vitamins comprise: vitamin C; thiamin (vitamin B₁); riboflavin (vitamin B₂); niacin (vitamin B₃); pantothenic acid (vitamin B₅); pyridoxine (vitamin B₆); biotin (vitamin B₇); folic acid (vitamin B₉); and vitamin B₁₂; and
a natural sweetener;
wherein the beverage composition is further characterized in that it is substantially free of potassium, phosphorous and magnesium.

17. The beverage composition of claim 16, wherein the vitamin D is present in an amount of up to 20 mcg.

18. The beverage composition of claim 16, wherein the water soluble vitamins are present in the amounts of: vitamin C, up to 120 mg; thiamin, B₁, up to 3 mg; riboflavin, B₂, up to 3.4 mg; niacin, B₃, up to 40 mg; pantothenic acid, B₅, up to 20 mg; pyridoxine. B₆, up to 20 mg; folic acid, B₉, up to 1600 mcg; vitamin B₁₂, up to 12 mcg; and biotin, B₇, up to 600 mcg.

19. The beverage composition of claim 16, wherein the natural sweetener comprises stevia in an amount of up to 2 g.

20. The beverage composition of claim 16 further comprising omega-3 fatty acids in the amount of up to 300 mg, wherein the omega-3 fatty acids comprise a-linolenic acid (ALA), docosahexaenoic acid (DHA) and eicosapentaenoic acid (EPA).

21. The beverage composition of claim 16, wherein the composition is in powder form.
